(19) United States
(12) Patent Application Publication
(10) Pub. No.: US 2026/0306250 A1
(43) Pub. Date: Oct. 8, 2026

(54) Intraocular Lens Fixation Device

(71) Applicant: California LASIK & Eye, Inc., Sacramento, CA (US)

(72) Inventor: Bradley P. Barnett, Sacramento, CA (US)

(73) Assignee: California LASIK & Eye, Inc., Sacramento, CA (US)

(21) Appl. No.: 19/684,845

(22) Filed: May 21, 2026

Related U.S. Application Data
(63) Continuation of application No. 17/109,919, filed on Dec. 2, 2020.
(60) Provisional application No. 62/942,274, filed on Dec. 2, 2019.

(51) Int. Cl. A61F 2/16

(52) U.S. Cl. CPC A61F 2/16; A61F 2002/169; A61F 2002/16902; A61F 2002/16905

(57) ABSTRACT

Please replace the Abstract with the following amended paragraph:

An intraocular lens (IOL) fixation device is provided that has an IOL-supporting frame that can be compressed elongated or shortened within a delivery system and an array of arms tipped with T-shaped fixation elements that can pass through a sclera wall in a collapsed state to catch on an opposite surface of the sclera wall when in an expanded state to achieve fixation. A cannula, catheter, or needle delivery system can hold the fixation device in a constrained fashion, compressing the collapsible T-shaped ends in the collapsed until deployed from the delivery system. A grasper can pull the T-shaped fixation elements through the sclera wall in a collapsed state.

### CROSS-REFERENCE TO RELATED APPLICATIONS

The present invention claims the benefit of U.S. Provisional Application Ser. No. 62/942,274, filed Dec. 2, 2019, which is hereby incorporated by reference in its entirety, including any figures, tables, and drawings.

### BACKGROUND

Some types of tissue fixation can be difficult and/or time-consuming to complete, particularly when performing suturing. Suturing typically requires focused concentration and two-handed operation. Further, suturing is sometimes performed on bodily regions that have challenging physical constraints. For example, due to physical constraints of the intraocular cavity, it can be difficult to form knots with the sutures within the eye even though sutures may be needed to affix hardware or repair damaged structures. Because of this difficulty, specialist surgeons are often required to handle difficult procedures such as secondary intraocular lens implantation or reconstruction of the iris and, in some cases when inferior techniques are performed, further damage to the eye such as glaucoma and corneal failure may occur.

A simpler method of fixation would not only decrease operation time but would also increase safety and overall adoption of intraocular fixation techniques.

### BRIEF SUMMARY

Memory material fixation devices and fixation systems including a delivery system and memory material fixation device are described.

A memory material fixation device for tissue fixation can include a main body that can be compressed elongated or shortened within a delivery system; and a collapsible catch-shaped end that can pass through a material in a collapsed state to catch on an opposite surface of the material when in an expanded state to achieve fixation.

A delivery system for a memory material fixation device comprising a main body and a collapsible catch-shaped end can include a chamber configured to hold a memory material fixation device in a constrained fashion including compressing the collapsible catch-shaped end of the memory material fixation device in a collapsed state while the memory material fixation device is within the chamber.

A fixation belt for an intraocular lens can include a clear polymer elastic. In some cases, at least one memory material fixation device is pre-attached to the fixation belt such that the intraocular lens, the fixation belt, and the at least one memory material fixation device can be collapsed together in a delivery cartridge for insertion and placement within an eye.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

FIG. 1 illustrates operation of an example fixation system having a delivery system with a pre-loaded memory material fixation device.

FIGS. 2A-2F illustrate example memory material fixation devices.

FIG. 3 illustrates example fixation elements of a memory material fixation device.

FIGS. 4A-4C illustrate configurations of how a memory material fixation device may be compressed elongated or shortened within a delivery system.

FIG. 5 illustrates deployment of a memory material fixation device for intraocular fixation.

FIG. 6 illustrates an example application of a fixation system for suturing an iris defect.

FIG. 7 depicts a memory material fixation device deployed in a running fashion to close a skin defect.

FIGS. 8A-8D illustrate example applications and fixation designs of a fixation system for attaching implantable devices, treatment substance delivery systems or structures within the eye.

FIG. 9 illustrates another embodiment of an IOL and corresponding fixation designs.

FIG. 10 depicts further examples of hardware fixation with the disclosed memory material device.

FIG. 11A depicts a memory material fixation device with an incorporated receptacle.

FIG. 11B depicts an example embodiment of a memory material fixation device with a receptacle in an IOL deployment.

FIG. 12 depicts another embodiment of an IOL fixation design and fixation devices.

FIG. 13 shows additional examples of secondary fixation configurations.

FIGS. 14A-14E illustrate IOL fixation belts.

FIGS. 15-18 illustrate various delivery systems suitable for use with a memory material fixation device and fixation belt.

FIGS. 19A-19C show views of a guide ring for guiding deployment of a fixation system for attaching intraocular devices.

FIG. 20 shows a plot of fatigue and cyclic loading of 3D printed soft polymer for ophthalmic applications.

The figures depict various embodiments for purposes of illustration only. One skilled in the art will readily recognize from the following discussion that alternative embodiments of the structures and methods illustrated herein may be employed without departing from the principles described herein.

### DETAILED DESCRIPTION

The present disclosure provides an alternative to a traditional knotted suture by using an extensible memory material to achieve fixation. The disclosed devices and systems can be used for tissue repair.

In the field of ophthalmology, the disclosed devices and systems are suitable for iris defect repair, secondary intraocular lens implantation and other intraocular procedures that require suture fixation. In addition to applications in the field of ophthalmology, such devices and systems may have broader use in endoscopic surgery and other minimally invasive procedures.

As previously mentioned, when performing eye surgery or other types of eye repair, it is sometimes necessary for a practitioner to form sutures in the eye of the patient or affix implantable devices within the eye. Due to the inherent physical constraints of the intraocular cavity, forming knots within the eye to affix hardware or repair damaged structures can be difficult and time-consuming. In some cases, the suturing procedure may fall outside the purview of typical ophthalmological practice. The patient may instead be referred to eye surgeons who are skilled in specialized eye suturing techniques, or the ophthalmologist may elect to perform less difficult and possibly less effective procedures. For example, the use of anterior chamber intraocular lenses may be employed by ophthalmologists, compared to secondary intraocular lens implantation used by anterior segment surgeons. Anterior chamber intraocular lenses are known to have a risk of causing glaucoma and corneal failure.

Advantageously, a memory material fixation device, and associated fixation systems, can be used to provide a rapid and automatic fixation technique applicable to intraocular procedures. For example, it is possible to perform intraocular procedures through the sclera and avoid irritating, damaging, or otherwise risking eye health of a patient.

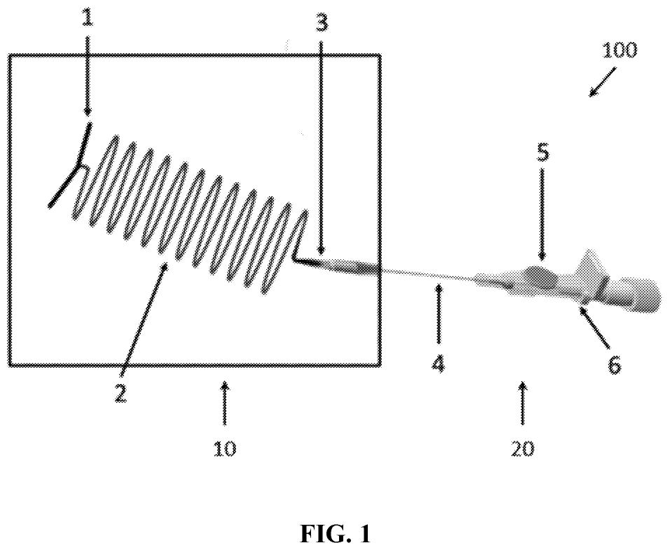

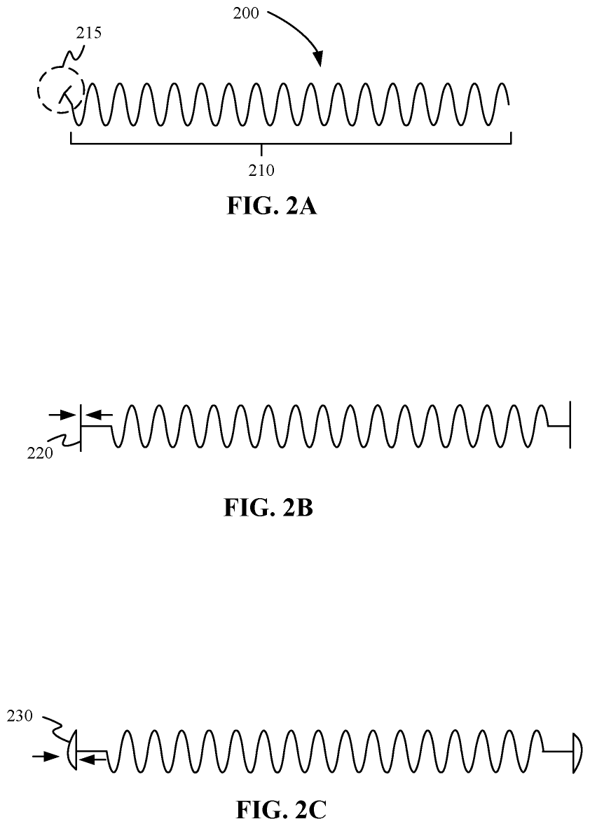

FIG. 1 illustrates operation of an example fixation system having a delivery system with a pre-loaded memory material fixation device. Referring to FIG. 1, a fixation system 100 can include a fixation device 10 and a delivery system 20. Fixation device 10 is magnified to show detail and is shown partially deployed from delivery system 20. Fixation device 10 comprises one or more fixation elements 1 as well as an elongate central portion 2 that interconnects the fixation elements. Here, a fixation element in the form of a collapsible catch-shaped end is shown. Fixation device 10 can be a memory material fixation device for tissue fixation, such as illustrated in FIGS. 2A-2E.

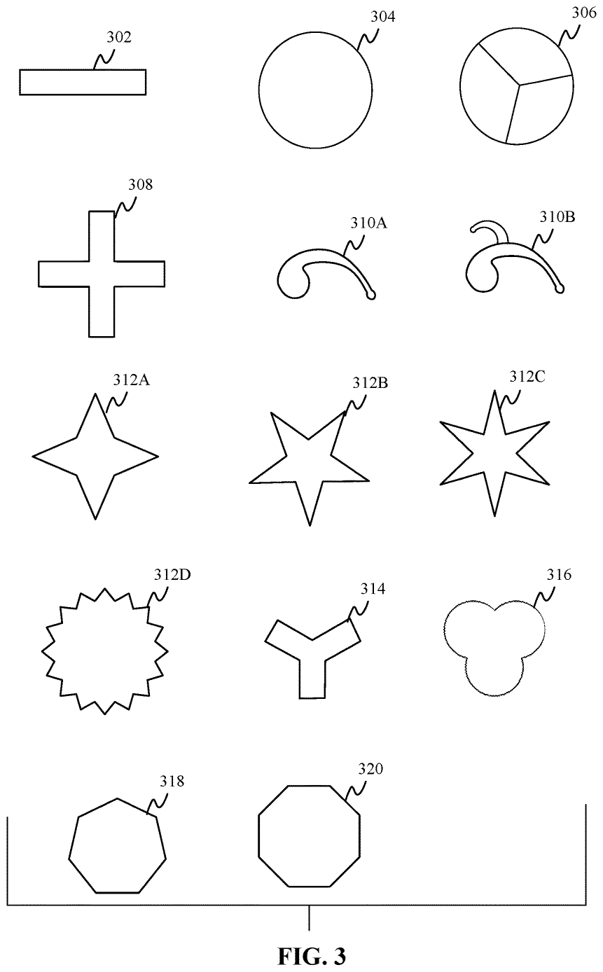

Delivery system 20 includes a body with a chamber configured to hold the fixation device 10 in a constrained fashion. The chamber can be implemented as a lumen of a needle 4; and fixation device 10 is held in confined state within the lumen of the needle 4 before it is deployed from the distal tip 3 of delivery system 20. The confined state can be a compressed elongated state or a compressed shortened state, such as described in more detail with respect to FIGS. 4A-4C.

Delivery system 20 can be a conventional delivery system, such as a catheter or needle. Several known strategies exist for deploying devices in a bodily system. In the simplest embodiment, fixation device 10 can be loaded in a microcatheter. Fixation device 10 can then be ejected with the pushing force of wire or other pushing element coaxially inserted proximal to fixation device 10 within the catheter. In some embodiments, a rigid needle can be incorporated on the tip of a flexible catheter, or the delivery system can be an entirely rigid device. A more advanced design includes the use of a retraction mechanism, where a needle is situated coaxially within a microcatheter. The device to be deployed lies within the lumen of the needle. When the needle retracts, the delivery system 20 deploys the fixation device 10. In some embodiments, similar to spring-based retraction mechanism employed in some angiocatheters, the delivery system utilizes a spring based or other mechanical retraction system to deliver the memory metal fixation element. Examples of needles that may be employed in a delivery system include, but are not limited to, ¼ circle, ⅜ circle, ½ circle (i.e., CT, CT-1, CT-2 and CT-3), ⅝ circle, compound curve, half curved (also known as a ski), half curved at both ends of a straight segment (also known as canoe).

Needles may also have a variety of point geometry including taper (needle body is round and tapers smoothly to a point); cutting (needle body is triangular and has a sharpened cutting edge on the inside curve); reverse cutting (cutting edge on the outside); trocar point or tapercut (needle body is round and tapered, but ends in a small triangular cutting point); blunt points for sewing friable tissues; and side cutting or spatula points (flat on top and bottom with a cutting edge along the front to one side) for eye surgery.

In certain instances, instead of the needle being part of the delivery system the needle may be on the end of the memory material fixation device. When part of the memory material fixation device, a needle may be permanently attached to the memory material fixation device or designed to come off the memory material fixation device with a sharp straight tug. Such a design is commonly used in suture designs with these “pop-offs” commonly used for interrupted sutures.

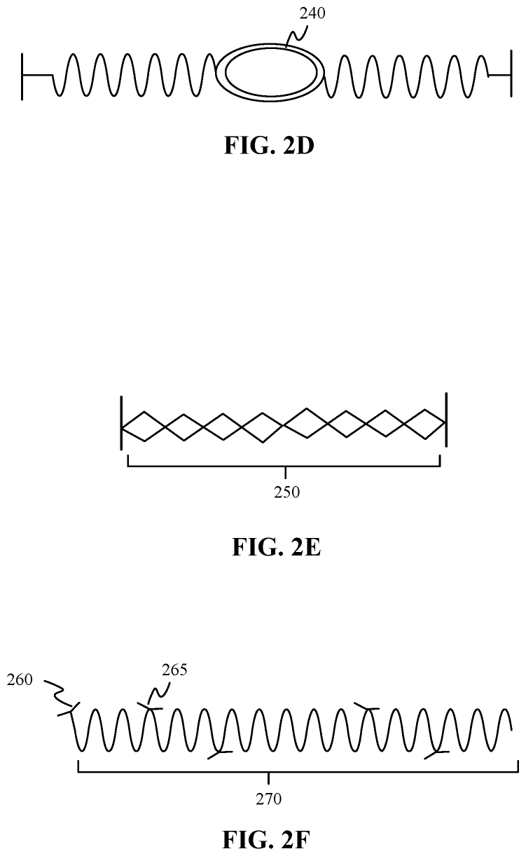

The design of fixation device 10 can vary depending on the surgical application. In the embodiment of FIG. 1, for example, fixation element 1 is in the form of a T-arm. However, other shapes such as shown in FIG. 3 may be used. In addition, central portion 2 can vary in length, width, and profile shape according to use. In a non-limiting example, central portion 2 can have a substantially three-dimensional helical or two-dimensional sinusoidal shape (e.g., a helical or sinusoidal profile). Other shapes are also possible, including straight, undulating, or sawtooth shapes. In general, central portion 2 can be designed to provide axial tension between opposing fixation elements and/or to draw together two sides of an area to be repaired.

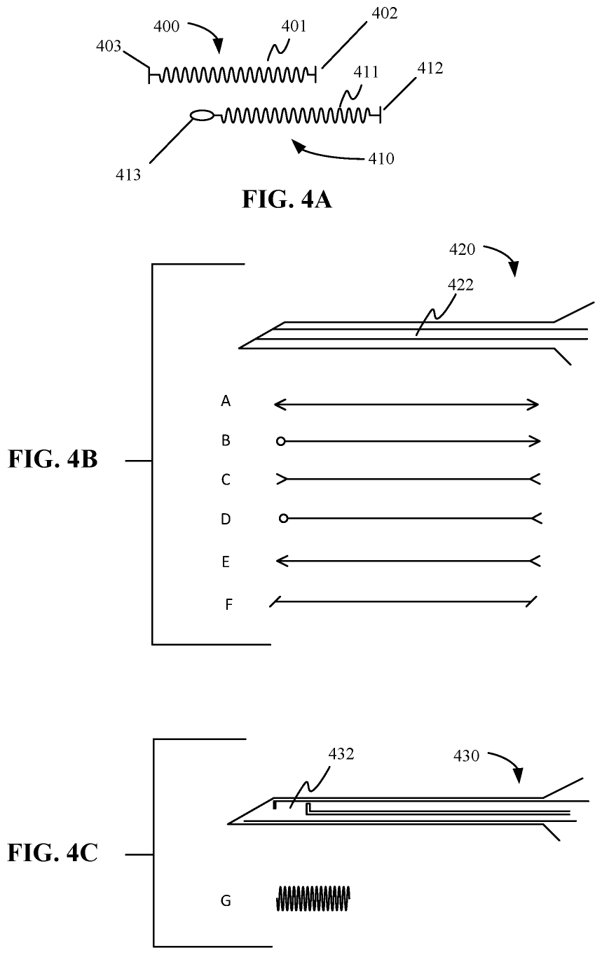

FIGS. 2A-2F illustrate example memory material fixation devices. FIG. 2A illustrates a representation of a memory material fixation device for tissue fixation. Referring to FIG. 2A, a memory material fixation device 200 can include a main body 210 that can be compressed elongated or shortened within a delivery system; and a fixation element 215 in the form of a collapsible catch-shaped end that can pass through a material in a collapsed state to catch on an opposite surface of the material when in an expanded state to achieve fixation (see e.g., FIGS. 4A-4C and 5).

As used herein, the term “memory material” can refer to any material that has the ability to return to an original shape after deformation. The mechanism for memory can be any of a variety of material properties. Examples can include many types of material, from shape-memory metal alloys to common molded plastic. Some examples of metal memory materials that could be used for a memory material fixation device include nitinol (nickel titanium), tantalum, platinum-iridium alloy, gold, magnesium, MP35N (35% cobalt, 35% nickel, 20% chromium, and 10% molybdenum), MP20N (50% cobalt, 20% nickel, 20% chromium, and 10% molybdenum) stainless steel, cobalt, nickel, chromium, molybdenum, titanium, copper-tin, copper-zinc, copper-zinc-tin, copper-zinc-xenon, copper-aluminum-nickel, copper-gold-zinc, gold-cadmium, gold-copper-zinc, iron beryllium (Fe3Be), iron platinum (Fe3Pt), indium-thallium, iron-manganese, iron-nickel-titanium-cobalt, nickel-titanium-vanadium, silver-cadmium, and combinations thereof.

Examples of polymeric memory materials that could be used for a memory material fixation device include polyvinyl acetate/polyvinylidinefluoride (PVAc/PVDF), blends of PVAc/PVDF/polymethylmethacrylate (PMMA), polyurethanes polynorbomene, polycaprolactone, polyenes, nylons, polycyclooctene (PCO), blends of PCO and styrene-butadiene rubber, styrene-butadiene copolymers, polyethylene, trans-isoprene, blends of polycaprolactone and n-butylacrylate, and combinations thereof.

The memory material fixation device 200 can thus be formed in a prescribed shape from a shape memory material of the metal and/or polymeric memory materials.

The cross-section of the memory material fixation device 200 may be circular, rectangular, oval, triangular or any other shape. The cross-sectional diameter of the main body 210 may be, for example, approximately 10 to 70 μm or any diameter. The main body 210 may be a helical coil and can be fabricated by deforming a metal memory material about a mandrel or other object, brought to the transformation temperature for that memory material to set it to the new shape, and then removed from the mandrel. For polymeric memory materials the memory material fixation device may be 3D printed, may be casted, or may be formed through other means known in the art. For example, memory material may be wound over a spherical mold having a particular diameter. The memory material may then be heated or cooled such that it plastically or otherwise deforms to the shape of the mold. Following this deformation, the material can then be returned to standard ambient temperature all the while retraining its new shape.

When not in its confined state, a memory material fixation device may compromise sections that are not linear. Examples of non-linear structures that may enable a degree of accommodation of various lengths include a helix and other coiled structures. Similar to a spring, such a shape inherently contracts and expands depending upon the longitudinal force applied. This coiled structure can also act to fixate the memory material fixation device in the tissue or device through which it is passed. This fixation is akin to a screw in wood. For ocular applications the resistance of a coiled memory material fixation device is sufficient to allow for any slack or redundancy in length of the fixation element to be reduced while not applying undue forces on the delicate structures of the eye that could cause “cheesewiring”, or tearing of the fixation element through the delicate structures. Such a design is superior to the current state of the art in which a suture is looped and tied through a secondary intraocular lens.

The disclosed design is unlike a suture that has no ability to reduce in length to compensate for slack. This slack in a suture is one of the main causes of extrusion of the suture through the conjunctiva as it mechanically abrades through the conjunctiva almost like a band-saw. When the suture rotates and the knot is exposed, this exposed knot very commonly leads to breakdown of the overlying conjunctiva. This acts as a nidus for intraocular infection. Accordingly, implementations of the fixation element of the memory material fixation device used for intraocular fixation is configured to be relatively flat and free of rough edges in order to not create a frictional force that damages adjacent tissues.

In various implementations, an outer surface of the collapsible catch-shaped end (e.g., for fixation element 215) is configured to minimize tissue erosion, for example by maintaining a flat or rounded profile. In certain designs, instead of a purely linear shape or ends, the fixation element can be in a form of a loop or petal-like structure or structures in which no wire tip is present. In other instances, the memory material element can be coated with PTFE or another biocompatible polymer to decrease trauma to adjacent structures. Potentially another means of overcoming the trauma of the fixation element is to use a small patch graft made of PTFE or another synthetic material or made of irradiated cornea, sclera or other biological patch graft.

FIG. 2B shows a memory material fixation device with a low-profile fixation element 220. FIG. 2C shows a memory material fixation device with a protruding (but smooth) fixation element 230. FIG. 2D shows a memory material fixation device with an integrated receptacle/frame 240. The integrated receptacle/frame 240 may be used to hold an implantable device and/or treatment delivery system. FIG. 2E shows a memory material fixation device with a lattice pattern body 250. FIG. 2F shows a memory material fixation device with a fixation element 260 at an end and multiple fixation elements 265 interspersed throughout the main body 270.

The length, number of coils or bends, and even the shapes of the ends may vary depending on implementation. In some cases, a fixation device can have one collapsible catch-shaped end of one shape and/or size and another collapsible catch-shaped end of another shape and/or size.

FIG. 3 illustrates example fixation elements of a memory material fixation device. As shown in FIG. 3, a fixation element can be in the form of a T-end 302, disc 304, cone 306, cross 308, barb 310A, 310B, star 312A, 312B, 312C, 312D, Y-end 314, trefoil 316, hexagon 318, octagon 320, as examples.

Much like surgical suture currently employed the memory material of a memory material fixation device can be deployed in a permanent manner or may be broken down by the body. Examples of materials that are degraded by the body include non-cross-linked collagen, cross-linked collagen, 90% glycolide and 10% L-lactide (vicryl), polyglycolic acid, polylactic acid, monocryl, polydioxanoe. Non-absorbable materials that be incorporated include nylon, polyester, PVDF, polypropolene and a variety of other polymers. For permanent applications, the memory material can be nitinol or another metal alloy or, alternatively, the memory material fixation device may be comprised of thermoplastic polymers. In other instances, the material of the memory material fixation device is responsive to an electromagnetic current, such that when exposed to an electromagnetic current the material changes configuration. The memory material of the fixation device can be monofilament or braided filaments may be used.

In certain instances, the memory material fixation device includes both components that are formed of memory material and components that are not formed of memory material. For example, in one implementation, a memory material fixation device can include a nylon component that extends from a memory material component such as a nitinol T-arm. In this example implementation, the nylon component can be used to adjoin the nitinol T-arm to another element.

Returning to FIG. 1, in some cases, the delivery system 20 includes a hub 6 that allows for retraction of the body having the chamber (e.g., needle 4) within its housing. An actuator 5 is used to pull the needle into the hub 6 of delivery system 20, thereby deploying fixation device 10. Actuator 5 can be, for example, a spring-based retraction mechanism. To preclude the migration of the fixation memory material element into the hub of the device along with the catheter or needle, the catheter or needle can be coaxially threaded over a pusher rod that occupies the lumen of the delivery catheter thereby displacing the memory material element distal to the catheter. In some cases, the delivery system 20 includes a plunger that pushes the fixation device 10 from the chamber out the distal tip 3 of the needle 4, thereby deploying the fixation device 10. The plunger can be coaxial to a lumen of the needle 4.

In some embodiments, the method of withdrawing the needle, cannula, or catheter into the handle of the delivery system involves compressing a spring disposed in the needle so that, with the catheter or needle in its extended version, the needle is maximally compressed. The system is held with this potential energy until the end-user pushes a lever or otherwise relieves an element preventing expansion of the spring. Once deployed the spring is free to expand thereby pulling the catheter or needle into the handle assembly. In an alternate design the catheter or needle can be manually withdrawn into the delivery handle with simply a tab that connects to the catheter or needle that can be pulled by the end user back within the handle thereby manually withdrawing the element. Alternatively, the device may be engineered to provide a pneumatic, electric or mechanical mechanism of conventional design for either driving the delivery catheter or needle into the handle or forcing the memory fixation element out of the delivery system. These forces can be controlled by a manual switch on the device or through a remote location such as a foot switch. In some cases, the switch is mechanical and has a compression spring telescopically disposed over the delivery catheter or needle. When the memory material fixation device is within the delivery system, the memory material fixation device is precluded from falling out of the delivery catheter or needle since the memory material fixation device maximally expands within the delivery needle or catheter thereby holding itself through friction within the lumen. As the catheter or needle retracts, the memory material fixation device reaches or nears the proximal end of the housing and is stopped from withdrawing with the catheter or needle. In such a design, the maximal possible compression of spring prevents the distal end base of the catheter or needle from ever reaching the most distal point of the handle housing. This in turn provides a means of controlled disposition of the memory material fixation device from the housing.

Accordingly, the memory material fixation device can be maintained in the confined state and be delivered from a lumen or channel of a cannula, needle, or catheter, as some examples.

In some cases, the delivery system can take the form of a capsular tension ring injector. This type of system is suitable for delivery of a memory material fixation device having a female or loop end (or even a temporary loop that a T-end or other grasping end shape can catch) in which a hook of the capsular tension ring injector can be inserted. The hook can then be drawn coaxially within the delivery system such that within the delivery system the hook cannot be released from the loop in the delivered device. Accordingly, a delivery system can be provided that includes a hook configured to releasably couple to a proximal end of the memory material fixation device; and a tensioner configured to draw the hook within the delivery system, thereby loading the memory material fixation device within the chamber.

Such a design is advantageous as it not only allows the end user to withdraw the device in and out of the catheter until appropriate placement for deployment, but it also enables the end user the ability to self-load the device into the delivery system. In certain instances it may be non-ideal to hold the memory material fixation device in a confined state for an extended period of time. If the device is loaded within the delivery catheter, the shelf-life of the product due to mechanical failure may be shortened. In addition, self-loading of the memory material fixation device by the end user enables a single delivery system that can be reused both within a single surgical case as well as after sterilization from case to case.

FIGS. 4A-4C illustrate configurations of how a memory material fixation device may be compressed elongated or shortened within a delivery system. FIG. 4A depicts a first memory material fixation device 400 with a main body 401 and two collapsible catch-shaped ends 402, 403 (in the form of T-arms) and a second memory material fixation device 410 with a main body 411 and one catch-shaped end 412 (in the form of a T-arm) and one loop shaped end 413. As illustrated in FIG. 4B, the delivery system 420 can include a chamber 422 for containing the memory material fixation device in a manner that constrains the fixation device causing elongation of the fixation device. Items A-F illustrate example positions of the fixation elements of the fixation device when elongated within a delivery system. Options A and B show the constraint of the fixation elements for devices 400 and 410 in delivery system chamber 422 causing inward positioning; options C and D show outward positioning of the fixation elements for devices 400 and 410; and options E and F show directional positioning. Upon deployment from the delivery system 420, the memory material fixation device contracts to a neutral position and the fixation element expands to take a neutral-stress shape to achieve fixation (e.g., as shown in FIG. 4A).

As illustrated in FIG. 4C, the delivery system 430 can include a spring-loaded chamber 432 for containing the memory material fixation device in a manner that constrains the fixation device causing compression of the fixation device. Item G illustrates an example compressed position of a fixation device when compressed within a delivery system. Upon deployment from the delivery system 430, the memory material fixation device expands to a neutral position and the fixation element expands to take a neutral-stress shape to achieve fixation (e.g., as shown in FIG. 4A).

FIG. 5 illustrates deployment of a memory material fixation device for intraocular fixation. In order to insert a memory material fixation device within the body, the memory material fixation device can be first held in a confined state to facilitate entry into a small incision or puncture site. In an ideal embodiment for secondary intraocular fixation to the sclera, a memory material fixation device is loaded within a needle. Similar to how ports for pars plana vitrectomy are placed without conjunctival peritomy, the overlying conjunctiva can be held in displaced position from the underlying sclera. Then, the needle for the delivery system can be advanced into appropriate location within the eye. The proximal portion of the fixation element can be passed through the intraocular lens and then the lagging element can be deployed between the sclera and the conjunctiva. When the needle is removed, the conjunctiva will naturally move to its native position thereby ensuring the fixation element and the needle hole in the conjunctiva are not directly aligned. For scleral fixated lens, it is ideal for all the fixation elements to be appropriately positioned in the X, Y and Z plane to ensure no lens tilt. To accomplish this an external guide can be applied on the ocular surface circumferentially around the cornea similar to a Failinger Ring but with two or more lumens through which the delivery system can pass coaxially through.

For example, referring to frame A, for operation within an eye 500 such as related to a lens 502, a delivery system 510 with a pre-loaded memory material fixation device (not shown) can be inserted through the sclera 506 via a puncture site 520. As reflected in FIG. 4B, the shape of the memory material fixation device can be roughly linear in its confined form within delivery system 510. As shown in frame B, the delivery system 510 can be inserted through a second puncture site 525 at a location for fixing an element to the eye at the sclera 506. Referring to frame C, the fixation element 530 of the memory material fixation device 540 catches on the outer surface of the sclera 506 as the memory material fixation device 540 is deployed and the delivery system 510 retracted/pulled out of the eye, Once deployed from the delivery system 510 the memory material fixation device 540 can return to its original shape (see also FIG. 4A). Materials for the delivery system can include the memory metals and polymers listed herein for the memory material fixation device.

Accordingly, a method of intraocular fixation to a structure can include traversing a target structure by a delivery system for a memory material fixation device that comprises a main body and a collapsible catch-shaped end; inserting at least a tip of the delivery system through a hole made in a material of the structure for attachment of the memory material fixation device; and deploying the collapsible catch-shaped end of the fixation device from the delivery system, whereupon the collapsible catch-shaped end of the fixation device expands to take its neutral-stress shape of the expanded state to achieve fixation.

The ideal structure of the memory material fixation devices as well as the delivery systems are largely dependent upon the mode of surgical axis and the tissues requiring fixation. It stands to reason that more delicate tissue will require memory material fixation devices that are finer in diameter and have a much lower Young's modulus and tensile strength. For ocular applications in which tissue is very delicate as compared to other body tissue a memory metal such as nitinol has a diameter in the range of 0.01 mm to 0.07 mm may be ideal. For other applications such as repair of skin lacerations, thicker memory material may be ideal. For example, skin closure may benefit from 0.05 mm to 1 mm diameter memory material elements. Such an approach of using larger diameter in relation to tensile strength of the tissue in which fixation is needed is well known in the art in the setting of various diameter sutures.

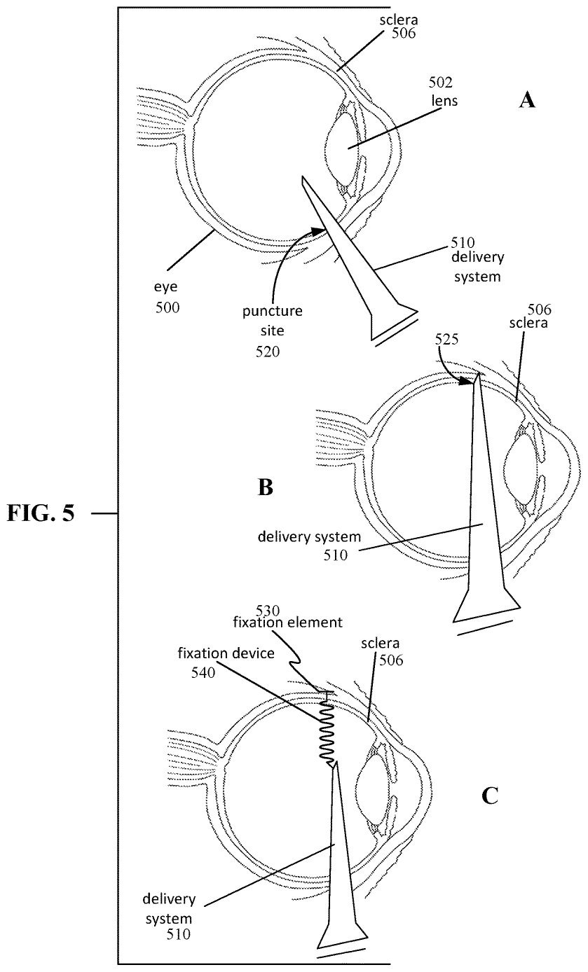

FIG. 6 illustrates an example application of a fixation system for suturing an iris defect. Here, the fixation device (e.g., 10), also referred to as a memory material fixation device (MMFD), is deployed in a running fashion to close an iris defect. A needle preloaded with the MMFD in a roughly linear confined state (A) is passed through alternating edges of the iris defect (B) until the needle with the MMFD held confined within its lumen has abutted the edges of the iris defect to each other. The needle is then retracted, allowing the MMFD to assume its unconfined state (C). The fixation element in the form of a T-arm acts to hold the MMFD at a precise location, while the central portion of the MMFD provides fixation. The MMFD, shown here with a helical shape, advantageously allows a degree of flexibility for mobile structures such as the iris. The helical structure also enables a degree of contraction and expansion so one device can be utilized for varying lengths of iris defect. Once deployed, the MMFD holds the iris defect closed (D). Not only are the described memory material fixation devices suitable for defects of the eye, the memory material fixation device is suitable for fixation of other tissues.

As will be recognized by a person of skill in the art, the fixation system described herein is not limited to use in the eye but may also be useful in replacing sutures or providing scaffolding in other regions of the body. For example, the device can be used for skin closure. Fixation devices such as the embodiments described hereinabove can be used for rapid closure of skin wounds in the field and in clinic. This may be particularly attractive for closure of wounds in military and remote settings. The fixation device can be placed to bridge tissue, and then cyanoacrylate glue can be applied in order to seal the wound.

In some embodiments the fixation device may have a plurality of fixation arms disposed along the length of the central portion. This can allow the closure of wounds of differing lengths. The device can be passed in a running fashion through a wound, and an intermediate fixation arms can expand along the length of the wound. Excess length and/or fixation arms can be trimmed. Alternatively, the device can have a sliding fixation arm that can utilize a loop or a ratcheting mechanism similar to a cable tie. In other embodiments, multiple shorter MMFDs could be deployed in an interrupted fashion.

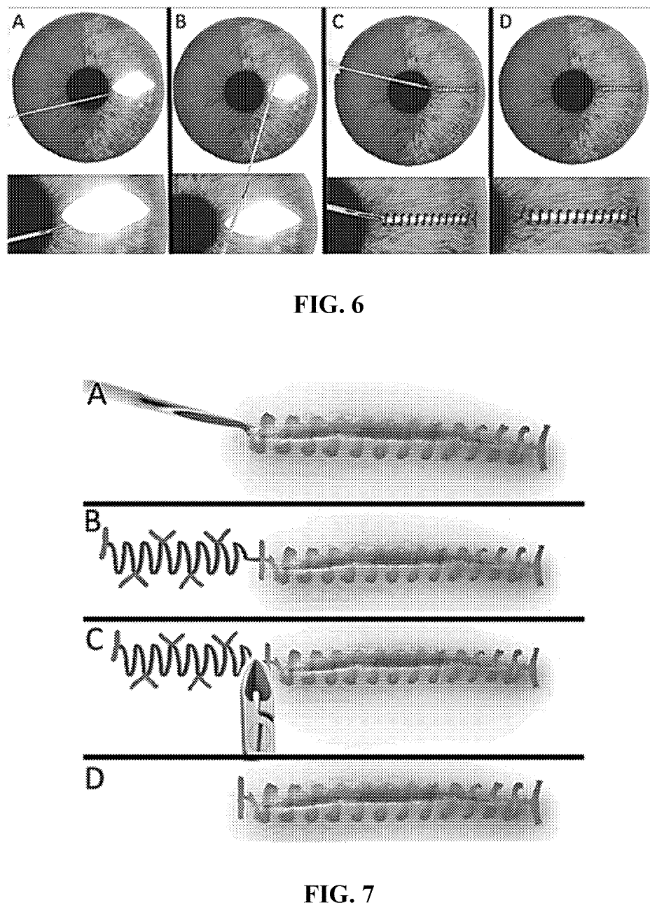

FIG. 7 depicts a memory material fixation device deployed in a running fashion to close a skin defect. Referring to frame (A), a needle preloaded with the memory material fixation device in a roughly linear confined state is passed through alternating edges of a skin defect until the needle with the MMFD held confined within its lumen has abutted the edges of the skin defect to each other. The needle is then retracted, thereby allowing the MMFD to assume its unconfined state, as illustrated in frame (B). Once deployed, the MMFD holds the skin defect closed, not unlike a running suture. The excess MMFD with a plurality of T fixation arms can be trimmed to the appropriate length as shown in frame (C), leaving a MMFD of the appropriate size to close the skin defect as shown in frame (D). Alternatively, the device may have a sliding fixation arm that can utilize a loop or a ratcheting mechanism similar to a cable tie.

In addition to suturing implementations, one or more MMFDs can also be used as a structure or scaffolding for the interior of the eye and can further be used in conjunction with implantable devices and/or treatment substance delivery systems.

In some cases, MMFDs can be delivered by inserting a delivery system into or through a sclera of an eye; deploying a distal fixation element of the MMFD device to a first region; navigating the delivery system to a second region; and deploying a proximal fixation element of the MMFD. The MMFD device can further be attached to an intraocular lens. In some cases, the fixation element of the MMFD is threaded through an intraocular lens. The first and second regions can correspond to an iris repair region. In some cases, MMFDs can be used to attach to and be deployed with an Ahmed capsular tension segment.

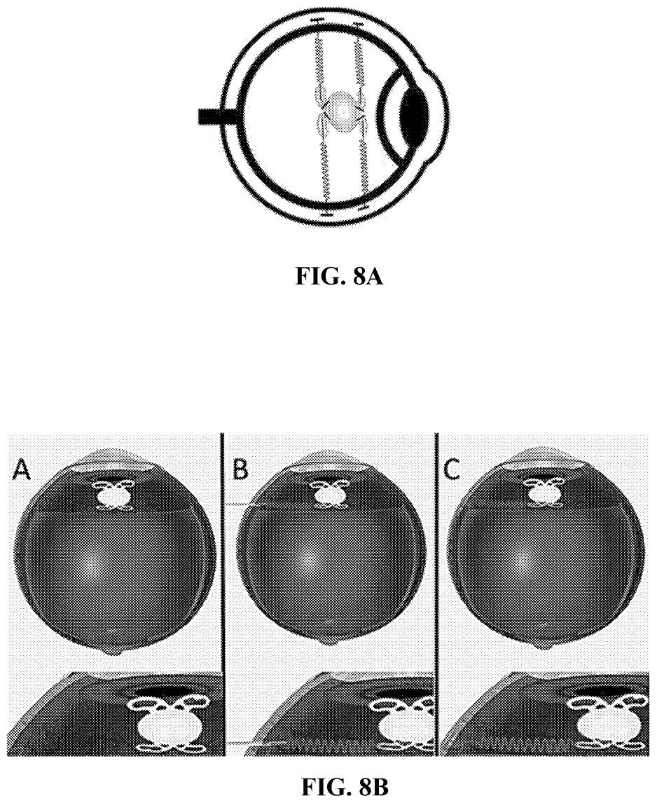

FIGS. 8A-8D illustrate example applications and fixation designs of a fixation system for attaching implantable devices, treatment substance delivery systems, or structures within the eye. Referring to FIG. 8A, a plurality of MMFDs is used for secondary intraocular lens (IOL) implantation. A MMFD is used to secure each of four quadrants of the intraocular lens. The T-arm fixation element affixes the MMFD to the IOL on one end, and to the scleral wall at the opposite end. The helical portion of the MMFD can be sized to act as a tension spring to hold the IOL taught. The extendable nature of the central portion also enables an element of a single length to be used in a range of eye sizes while an acceptable spring force is maintained. In addition to T-arm fixation for fixation to the sclera, one or more fixation elements may be present to hold the MMFD anchored within the wall of the sclera.

Referring to FIG. 8B, steps A, B, and C may be performed to attach an implantable device of an intraocular lens. In step A, an intraocular lens is inserted into the eye in a conventional manner. In step B, similar to the technique for scleral sutured intraocular lens, a needle is passed through the sclera. Unlike scleral sutured lenses that require a limited conjunctival peritomy, the MMFD can be delivered without conjunctival peritomy. The tip of the delivery system is inserted through one or more holes in the intraocular lens and the distal fixation element is deployed. In step C, the needle of the delivery system is further retracted until the proximal fixation element opens beneath the conjunctiva, but within or outside of the scleral wall (C). Alternately, in some cases limited peritomy with a scleral window may be preferable, and then the proximal fixation element is opened within the scleral window pocket. The scleral window is then glued or sutured into place and the conjunctiva is closed.

In the examples shown in FIGS. 8A and 8B, each quadrant (i.e., tab or corner) of an IOL is secured by one MMFD. However, other fixation configurations are possible.

FIG. 8C depicts an array of possible alternate secondary IOL fixation designs. One or more for fixation device (e.g., 10) can be interwoven in a variety of configurations (e.g., as shown in frames A-F) to achieve IOL fixation. As can be seen, it is also possible to pass a single MMFD through multiple quadrants. For strategies that employ a MMFD that passes through the optical center of a lens, optically transparent materials can be incorporated in the MMFD to prevent the fixation from creating a visual distortion. In these cases, the MMFD delivery system is inserted first in sclera, then passed through the holes in the IOL, and then passed through the sclera. In some embodiments the exit location of the MMFD is approximately 180 degrees from the entry point. The distal fixation element of the fixation device is then deployed and the delivery system needle is retracted back through the holes of the IOL all the way back to the initial entry point in the sclera. In a final step, the proximal fixation element is deployed.

FIG. 8D depicts a secondary IOL fixation design for iris fixation. Here, in frame (A), an IOL is delivered behind the iris. An MMFD, which is held constrained within a delivery system needle, is passed from the anterior segment through the iris and into a receiving portion of the IOL. The distal fixation element is deployed behind the IOL, and the proximal T-arm fixation element is deployed in front of the iris, as illustrated in frame (B). This approach can be repeated for each of the four quadrants of the IOL. Alternatively, as illustrated in frame (C), a single MMFD can be used to anchor two quadrants of the IOL by passing the delivery system through the iris and then through a first hole on one side of the IOL, followed by a second hole on the other side of the IOL, followed by passage of the delivery system through the iris again. The delivery system is then retracted, thereby deploying both T fixation elements in the anterior segment in front of the iris.

As will be obvious to one skilled in the art, needles of various shape and deployment devices of variable flexibility will be necessary in order to make these surgical maneuvers. It is further to be understood that the fixation system can also be used in cooperation with other surgical assistance devices. For example, the fixation device can be used with an endoscopic device in order to visualize structures behind the iris.

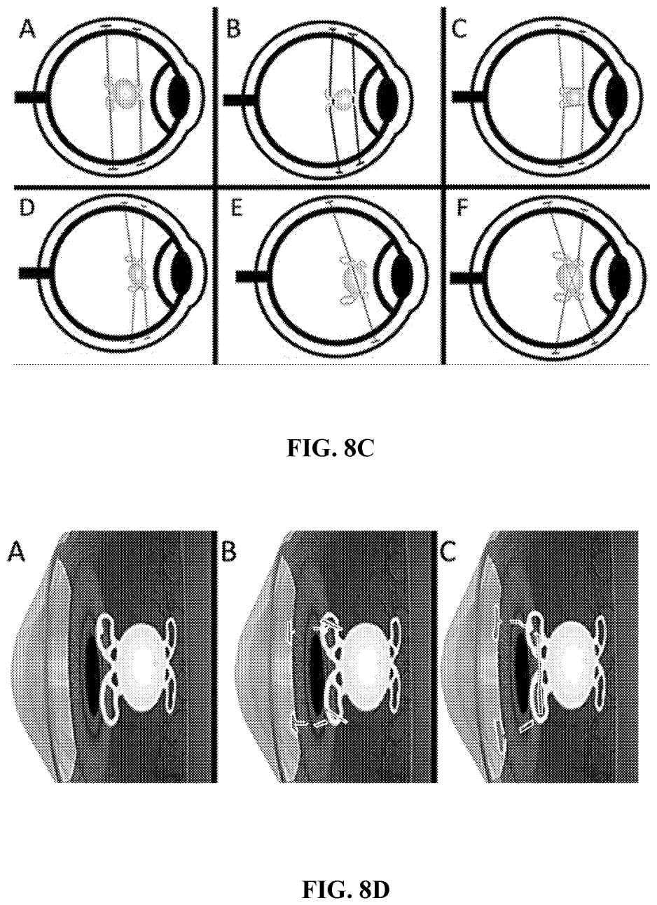

FIG. 9 illustrates another embodiment of an IOL and corresponding fixation designs. Referring to FIG. 9, some types of IOLs do not have a hole through which a fixation can be passed. In instances where there is a protrusion on the IOL, such as shown in frame (A), a memory material fixation device with a loop or female end can be used. That is, a memory material fixation device can have a collapsible catch-shaped end and a female end. The female end may be formed from a loop knot such as a slipknot or noose.

For example, with reference to frame (B), an IOL 900 such as shown in frame (A) can be inserted and then the MMFD with a loop 902 on its distal end may be advanced through the wall of the sclera. The loop is then looped around the placement arm, or haptic 905, of the IOL 900. A tension force is exerted on the fixation device, thereby cinching the knot around the IOL. The delivery system is then pulled back to allow the fixation element to open in the wall of the sclera.

In an alternate embodiment, such as shown in frame (C), a single loop 902 can be attached to two central portions 906A, 906B with corresponding collapsible catch-shaped ends 908A, 908B in a “Y” shape to attach the IOL to the sclera.

In still another embodiment, two MMFDs-one with a loop fixation element and a T-arm fixation element can be used in combination, such as shown in frame (D). The MMFD 910 with the loop element 912 is first placed around the haptic of the IOL. The needle for the MMFD 914 with T-arm fixation element 916 is passed through the loop element 912 and deployed so it opens through the loop element 912 at the distal end D1 and in the sclera wall at the proximal end P1. The loop element 912 is then pulled tight, cinching the loop 912 around both the IOL haptic (e.g., 905) and T-arm 916. The proximal end P2 of the MMFD with the loop element (which can be of a different type, such as a T-arm) is then deployed in the scleral wall.

As can be seen in the above examples, a method of intraocular fixation can include traversing the eye through the sclera by a delivery system for a memory material fixation device that comprises a main body and a collapsible catch-shaped end; and inserting at least a tip of the delivery system through a hole made in the sclera at an opposite end from entry (e.g., beneath the conjunctiva) for attachment of the memory material fixation device. The method can continue by retreating through the target structure while deploying the main body of the memory material fixation device. This can include navigating the delivery system to a second region; and deploying a proximal fixation element of the memory material fixation device. In some cases, the original traversal can be through one or more holes in the intraocular lens such that the proximal fixation element can be attached to the intraocular lens when retreated through the one or more holes in the intraocular lens. In some cases, such as when there are no holes in the intraocular lens (or when the holes are not originally traversed), the proximal fixation element can be later attached to the intraocular lens, for example by threading the proximal fixation element through the intraocular lens.

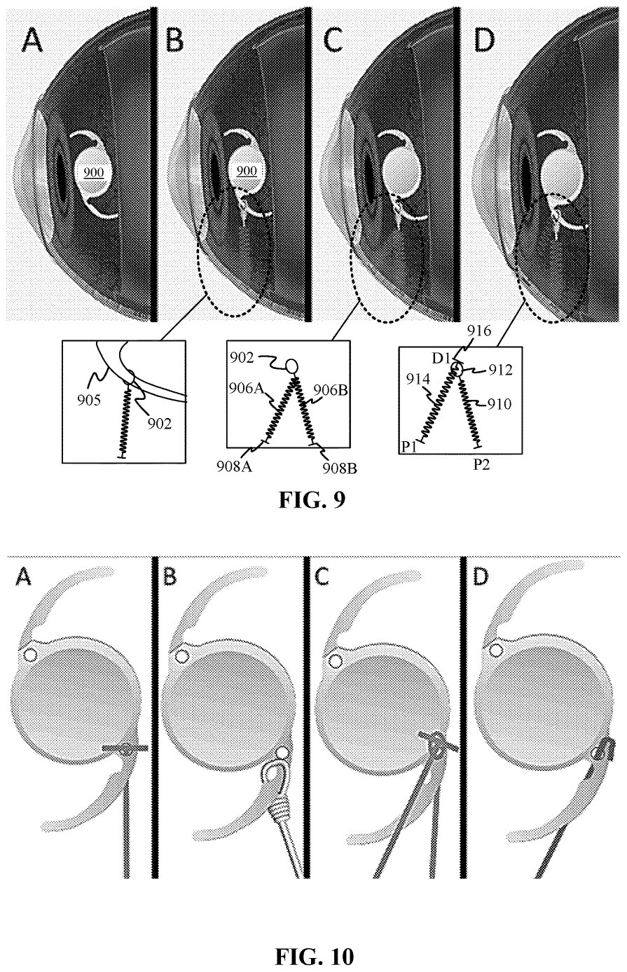

FIG. 10 depicts further examples of hardware fixation with the disclosed memory material device. For example, existing hardware features can be used to accommodate a T-arm fixation element (A), a noose or slip-knot fixation element (B), a combination of T-arm fixation element and a loop fixation element (C), and a hook fixation element (D). Numerous other fixation strategies can be envisioned without departing from the scope of the disclosure.

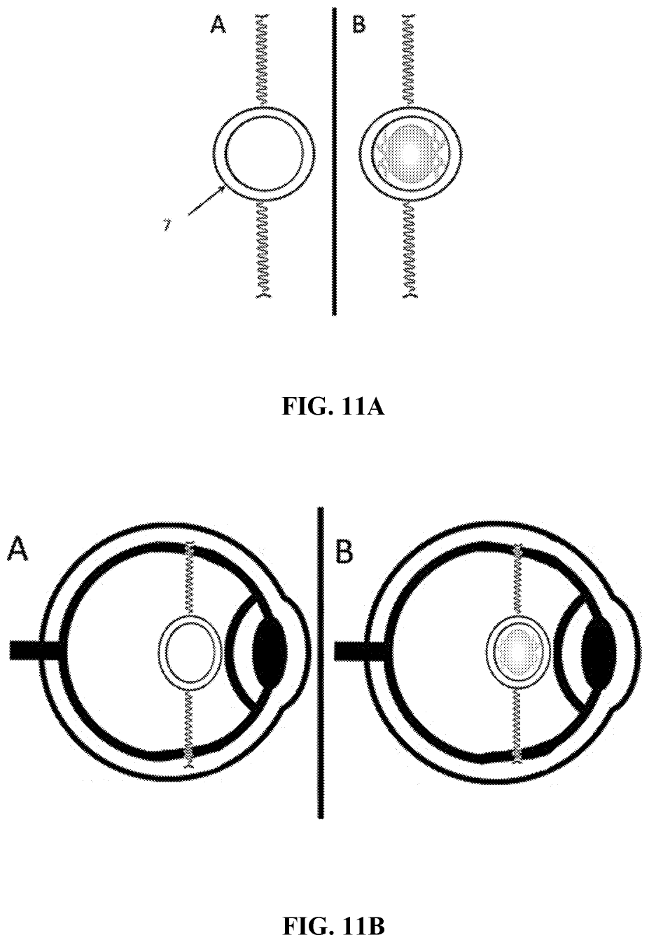

FIG. 11A depicts a memory material fixation device with an incorporated receptacle. Referring to FIG. 11A, a memory material fixation device with incorporated receptacle, or frame 7, is provided to hold depot drug delivery vehicles, intraocular lenses, intraocular pressure sensing devices, fiducials, radioactive plaques, etc. In some embodiments, rather than using a fixation element to attach the fixation device to an IOL, frame 7 can be used to hold the IOL in place. Frame 7 can be, for example, in the form of one or more polymeric sheets and/or a semi-rigid frame. In a non-limiting example, a frame can have a donut structure that the IOL can be housed within, thereby holding the haptics within the lumen of the donut-like structure. This receptacle can be of any suitable size or shape in order to hold different objects. In addition, multiple fixation elements can emerge from a single fixation donut or can be secondarily added by incorporating a hole or other fixation point within the fixation donut.

In some embodiments, the frame 7 is formed of a memory material and comprises or is coated by a biocompatible polymer. For example, the use of PTFE affixed to nitinol is known in intravascular stent design. In some embodiments frame 7 is coated in a biocompatible polymer. In other instances this biocompatible polymeric sheet is affixed to but extends beyond the frame to create a receptacle for the deployment of other devices such as depot drug delivery vehicles, intraocular lenses, intraocular pressure sensing devices, fiducials, radioactive plaques, or any other device or substance to be affixed within the intraocular cavity. Examples of polymeric sheet materials that can be used include polyesters, such as poly(ethylene terephthalate), polylactide, polyglycolide and copolymers thereof; fluorinated polymers, such as PTFE, expanded PTFE and poly(vinylidene fluoride); polysiloxanes, including polydimethγl siloxane; and polyurethanes, including polyetherurethanes, polyurethane ureas, polyetherurethane ureas, polyurethanes containing carbonate linkages and polyurethanes containing siloxane segments. Any polymer that may be formed into a porous sheet can be used in this device provided the final porous material is biocompatible. Polymers that can be formed into a porous sheet include polyolefins, polyacrylonitrile, nylons, polyaramids and polysulfones, in addition to polyesters, fluorinated polymers, polysiloxanes and polyurethanes as listed above. In some embodiments, surface modification of the polymeric sheet can be performed to enhance biocompatibility.

FIG. 11B depicts an example embodiment of a memory material fixation device with a receptacle in an IOL deployment. Similar to designs described above, the MMFD, including the receptacle, is held in a confined fashion within a delivery system. The memory material fixation device is passed through an entry point of the sclera and then exits at an approximately opposite point on the eye. The distal fixation element is deployed and as the delivery needle retracts the donut receptacle is released from the needle and expands into its neutral shape. The fixation element is then released in the proximal sclera as shown in Frame (A). Next, the IOL is deployed within the fixation donut. The haptics of the IOL expand into the negative space within the fixation donut to hold the IOL securely in place as shown in Frame (B).

As can be seen by the various examples, a method of intraocular fixation to a structure can include traversing a target structure by a delivery system for a memory material fixation device that comprises a main body and a collapsible catch-shaped end; inserting at least a tip of the delivery system through a hole made in a material of the structure for attachment of the memory material fixation device; and deploying the collapsible catch-shaped end of the fixation device from the delivery system, whereupon the collapsible catch-shaped end of the fixation device expands to take a neutral-stress shape to achieve fixation.

In a further implementation, the delivery system is inserted through a hole made in a sclera of an eye. In some cases, the hole made in the sclera of the eye is at a position indicated by a guide ring.

The method above can further include retreating through the target structure while deploying the main body of the memory material fixation device. In some cases, retreating through the target structure while deploying the main body of the memory material fixation device comprises: navigating the delivery system to a second region; and deploying a proximal fixation element of the memory material fixation device. Deploying the proximal fixation element of the memory material fixation device can include attaching the proximal fixation element to an intraocular lens. In some cases, deploying the proximal fixation element of the memory material fixation device comprises threading the proximal fixation element through an intraocular lens.

The proximal fixation element deployed at the second region can have a second collapsible catch-shaped end, a loop, or some other shape.

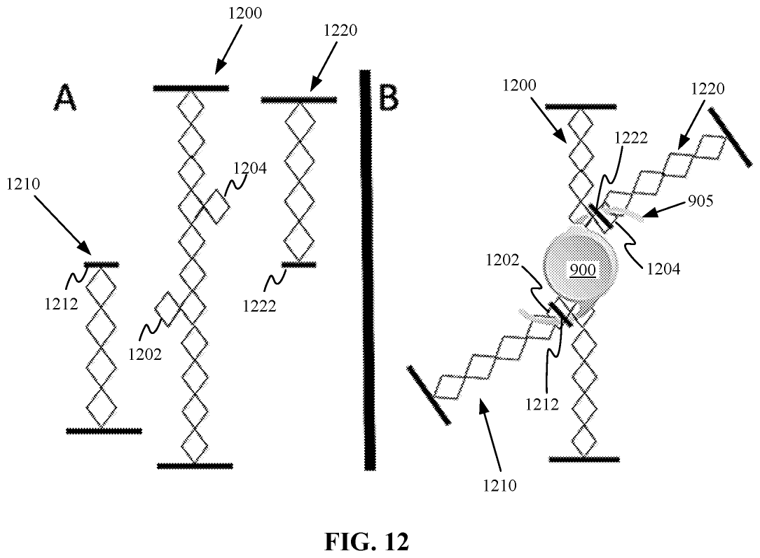

FIG. 12 depicts another embodiment of an IOL fixation design and fixation devices. Referring to FIG. 12, as shown in frame A and B, a primary fixation device 1200 with a lattice pattern main body includes a leaf extension through which a haptic 905 of an IOL 900 such as described with respect to FIG. 9 can be inserted. Of course, the illustrated IOL fixation design would also work with IOLs that have apertures. In addition, more or fewer leaf extensions may be provided. Here, the primary fixation device 1200 includes a first leaf extension 1202 and a second leaf extension 1204, positioned such that one haptic of the IOL can fit into one of the leaf extensions and another haptic of the IOL can fit into the other. The length and even the size of the weave of the section of the main body between the two leaf extensions can vary depending on implementation.

Rotation of the IOL is inhibited by the inclusion of at least one additional fixation device. The at least one additional fixation device attaches to the IOL via the same leaf extension through which a haptic is inserted. For example, a first fixation device 1210 can be deployed through the first leaf extension 1202 so the collapsible catch-shaped end 1212 (e.g., T arm) catches between the haptic arm and the first leaf extension 1202. In addition, a second fixation device 1220 can be deployed through the second leaf extension 1204 so the collapsible catch-shaped end 1222 (e.g., T arm) catches between the haptic arm and the second leaf extension 1204. Although a device with a lattice pattern main body is shown, the fixation devices can have other shapes, such as helical and sinusoidal.

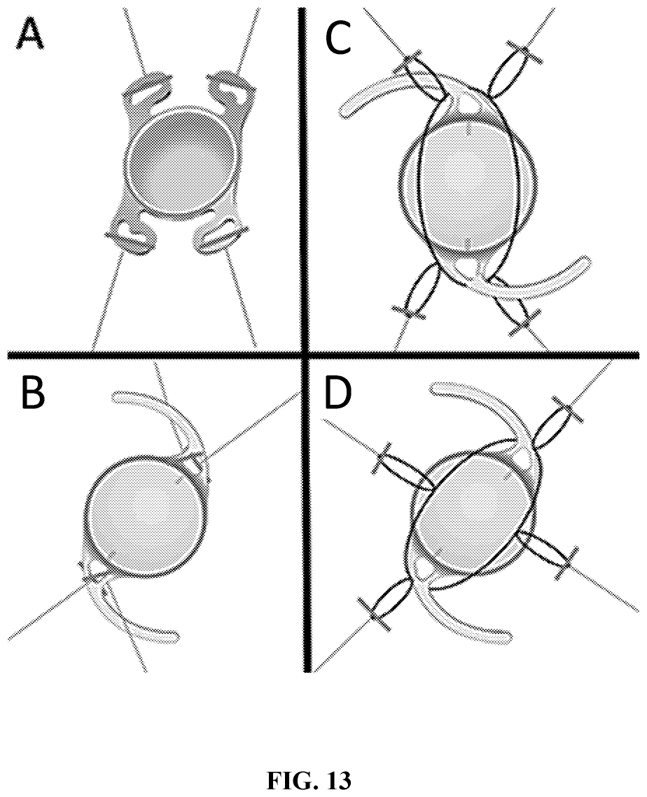

FIG. 13 shows additional examples of secondary fixation configurations. Frame A shows four T-arm fixation devices deployed through corresponding eyelets in an AKREOS IOL; and Frame B shows four T-arm fixation devices deployed through the two eyelets in an IOL having eyelets in the haptics. Four point fixation is achieved in Frame B through double T fixation in each eyelet.

Frames C and D show two simple band designs that can be used for supporting fixation configurations that are not limited by the positioning of eyelets in IOL devices (and can be used for IOL devices that do not have eyelets). The simple band design includes a main band and four eyelet loops for receiving T-arm fixation devices. The bands may be formed of suture or may be pre-fabricated into the main band and eyelet loops. In Frame C, two eyelets are positioned near one haptic and two eyelets are positioned near the other haptic. In Frame D, the eyelets may be equidistant.

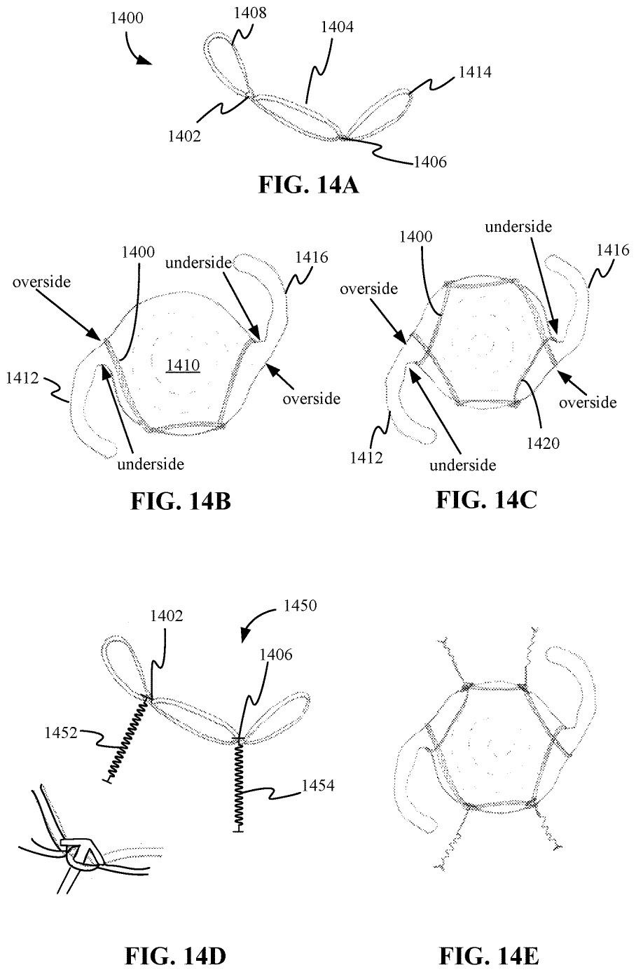

FIGS. 14A-14E illustrate IOL fixation belts. As previously mentioned, IOLs come in a variety of configurations, some with openings, but most without. Any lens can be converted into a secondary IOL through use of a fixation belt, such as illustrated in FIGS. 14A-14E.

Referring to FIGS. 14A and 14B, a memory material fixation device and fixation belt 1400 can include an elastic band formed of three segments by a first loop knot 1402 at one side of a middle 1404 of the three segments and a second loop knot 1406 at the other side of the middle 1404 of the three segments. A first segment 1408 defined by the first loop knot 1402 is configured to catch on an underside of a first haptic 1412 of an intraocular lens 1410 and a second segment 1414 defined by the second loop knot 1406 is configured to catch on an overside of a second haptic 1416 of the intraocular lens 1410.

Once the IOL 1410 with fixation belt 1400 is deployed in the bag of the eye, memory material fixation devices can be applied as previously described to attach to any suitable part of the elastic band, including via the loop knot (e.g., first loop knot 1402, second loop knot 1406) or simply between a segment of the band and the IOL.

In some cases, it can be desirable to have fixation in additional directions. FIG. 14C shows a further implementation that can support attachment by a memory material fixation device. Referring to FIG. 14C, a second elastic band 1420 can be included. As described with respect to fixation belt 1400, the second elastic band is formed with three corresponding segments by a corresponding first loop knot at one side of a corresponding middle of the three segments and a corresponding second loop knot at the other side of the corresponding middle of the three corresponding segments. A corresponding first segment defined by the corresponding first loop knot is configured to catch on an overside of the first haptic of the intraocular lens and a corresponding second segment defined by the corresponding second loop knot is configured to catch on an underside of the second haptic of the intraocular lens.

In some cases, instead of attaching the memory material fixation devices to the fixation belt after the IOL with fixation belt is deployed into the eye, a fixation belt 1450 such as shown in FIG. 14D can be used that further includes a first memory material fixation device 1452 for tissue fixation coupled to the elastic band via the first loop knot 1402 and a second memory material fixation device 1454 for tissue fixation coupled to the elastic band via the second loop knot 1406 (such as illustrated in enlarged inset). Referring to FIG. 14E, similar to the implementation described with respect to FIG. 14C, an IOL can have two fixation belts in the form of fixation belt 1450. Here, four memory material fixation devices are included; however, more or fewer may be used.

An elastic fixation belt can include a locking mechanism similar to a cable tie in which teeth engage a pawl in the head to form a ratchet so that as the free end is pulled the cable tie tightens and does not come undone. The belt may also include a tab that can be depressed to release the ratchet so that the belt can be loosened or removed. Accordingly, a fixation belt can include a cable tie connector on the first segment and/or the second segment for adjusting size of the elastic belt for fitting the intraocular lens.

The IOL fixation belt can be comprised of elastomeric biomaterials, such as silicone, PEEK, Polyethylene, PLA, Stainless steel, Titanium, cobalt chrome, thermoplastic elastomers, polyolefin and polydiene elastomers, poly(vinyl chloride), natural rubber, heparinized polymers, hydrogels, polypeptides elastomers or other biocompatible polymer or metallic alloy or combinations thereof.

For implementations having the memory material fixation devices attached to the fixation belt before deploying into the eye in a cartridge with the IOL, a delivery system can be configured with a grasper for grabbing and pulling the collapsible catch ends of the fixation devices back through the sclera. Indeed, such delivery systems are suitable for use with collapsible catch-shaped ends that are configured to be compressed in a grasper of a delivery system to pass through a material in a collapsed state before releasing to catch on an opposite surface of the material when in an expanded state to achieve fixation.

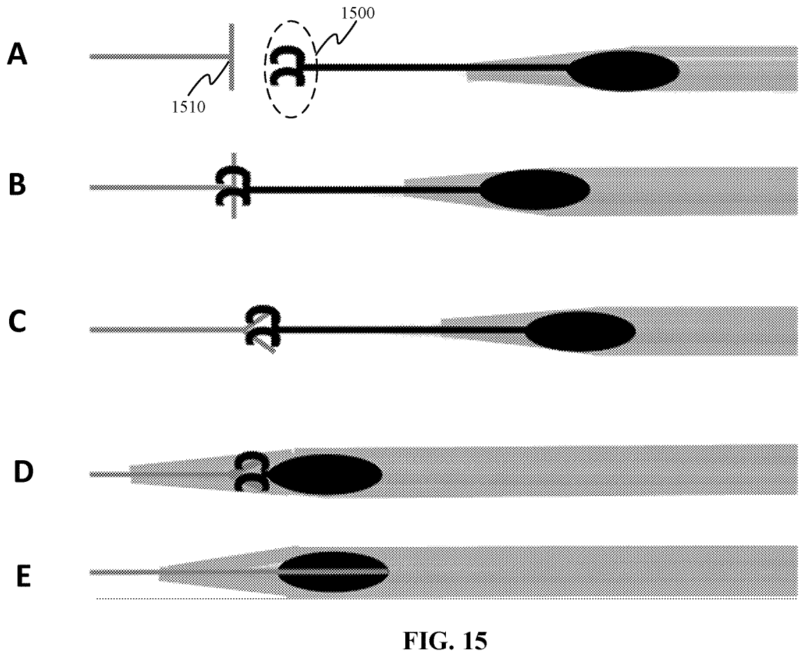

FIGS. 15-18 illustrate various delivery systems suitable for use with a memory material fixation device and fixation belt. Referring to FIG. 15, a delivery system can include a grasper 1500 having a double hook with a spacing between the hooks that is smaller than the diameter of a collapsible catch-shaped end 1510 of a memory material fixation device. As illustrated in sequence A-E, a delivery system with grasper 1500 can hook behind the end 1510 (as shown in B) to compress the end 1510 (as shown in C). The grasper 1500 is withdrawn back into the delivery system, bringing at least the end 1510 into a chamber of the delivery system, as shown in D and E.

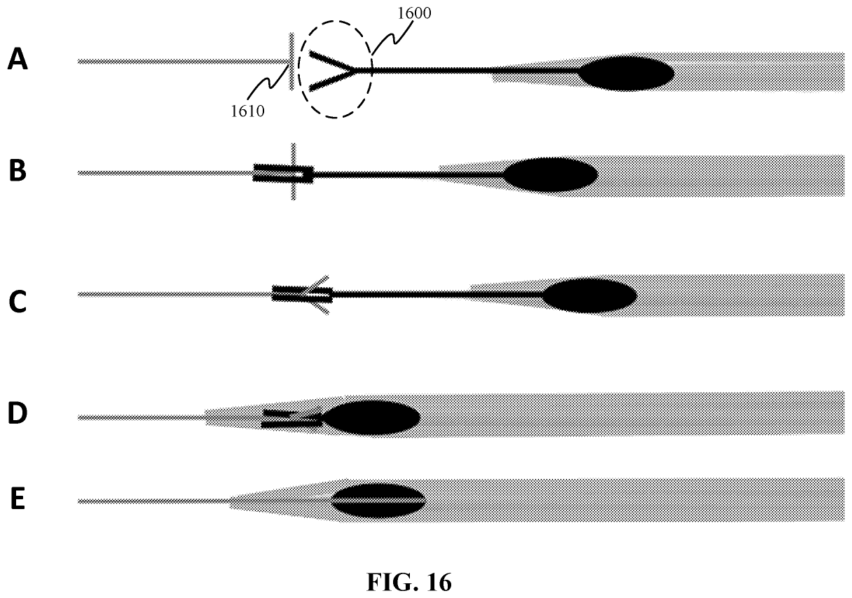

Referring to FIG. 16, a delivery system can include a grasper 1600 having a pincher end that grabs a collapsible catch-shaped end 1610 of a memory material fixation device. As illustrated in sequence A-E, a delivery system with grasper 1600 can grab the end 1610 (as shown in B) to compress the end 1610 (as shown in C). The grasper 1600 is withdrawn back into the delivery system, bringing at least the end 1610 into a chamber of the delivery system, as shown in D and E.

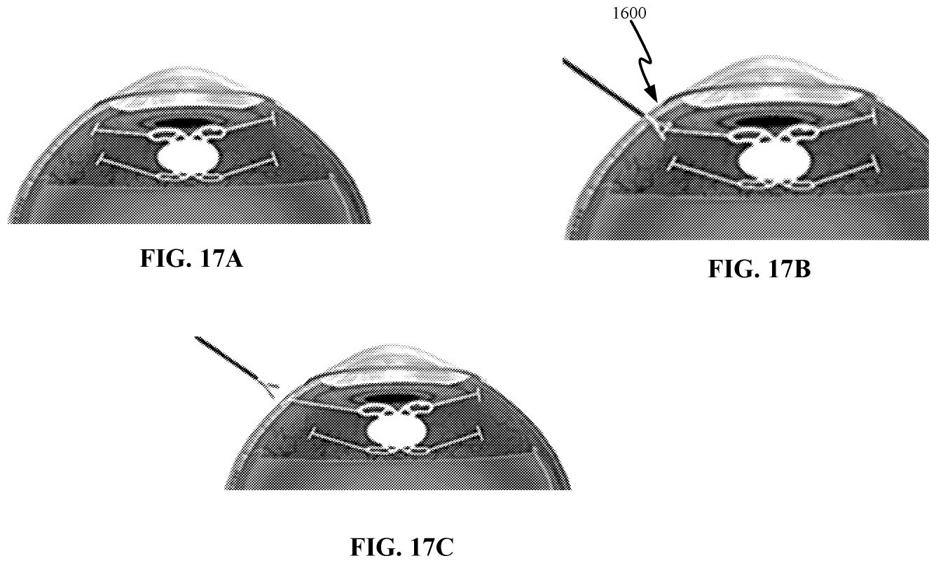

FIGS. 17A-17C show a diagram of the pull through technique in which fixation element already inside eye (FIG. 17A) and then the fixation element is pulled through the eye via delivery system 1600(as shown in FIGS. 17B and 17C).

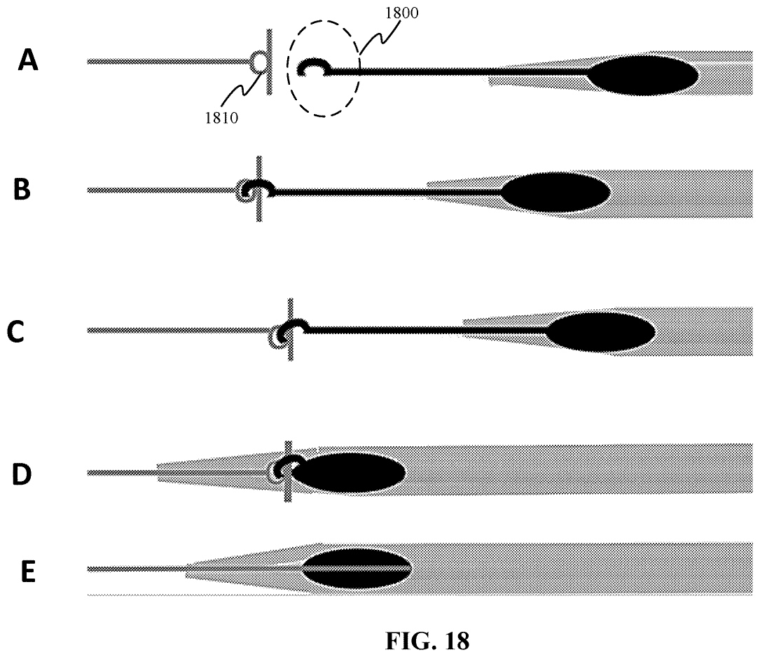

FIG. 18 shows a delivery system with a grasper 1800 with a single hook. The single hook shape is suitable for a collapsible catch-shaped end that has an eyelet 1810 or other shape that can be caught via the single hook. Here, the end with eyelet 1810 can be compressed once withdrawn into the delivery system as shown in E.

These various delivery systems could be used to preload the element and then deploy in eye as well as can be used to grab and then pull the element through the sclera as seen in FIGS. 17B and 17C.

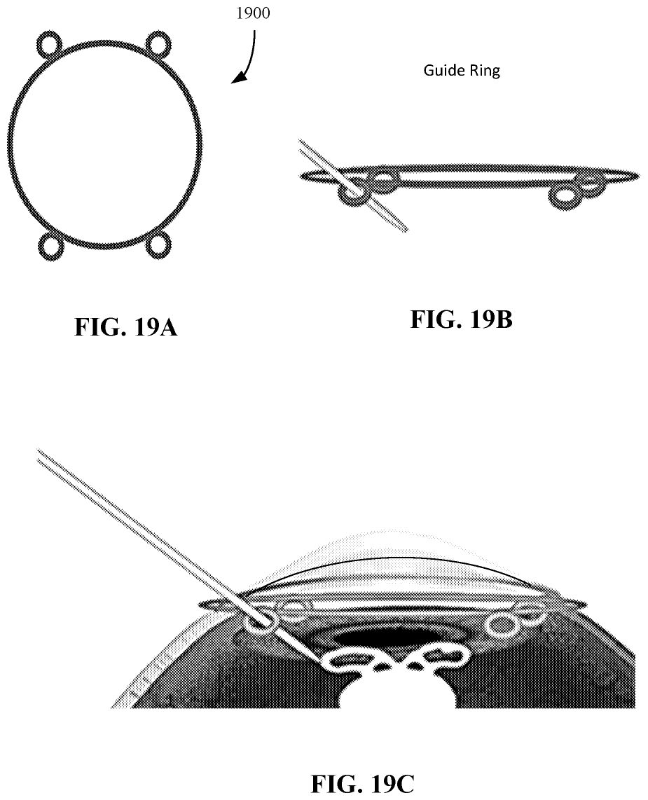

FIGS. 19A-19C show views of a guide ring for guiding deployment of a fixation system for attaching intraocular devices. Referring to FIGS. 19A-19C, a guide ring 1900 can be provided for assisting positioning of where insertion of a delivery system should be made. According to a specific implementation, the guide ring is approximately 12-15 mm in diameter made of a rigid material, including a rigid thermoplastic or metal such as stainless steel or titanium. The ring can be fitted with guide holes located at a precise distance from each other. These guide holes are utilized to ensure proper secondary IOL positioning and can be configured to assist with attaching IOLs according to any of the fixation designs illustrated herein. As illustrated in FIG. 19C, the ring guide can be simply placed equidistant from the limbus and manually held in place to ensure no change in motion. Alternatively, the ring can be sutured either through the guide holes or through separate fixation holes to suture the guide to the conjunctiva to restrain movement of the guide. In certain instances, the guide path can enable placement of vitrectomy ports at the precise equidistant locations. Such a guide may also be simply inked and used to stamp the conjunctiva with ink at the necessary locations for entry.

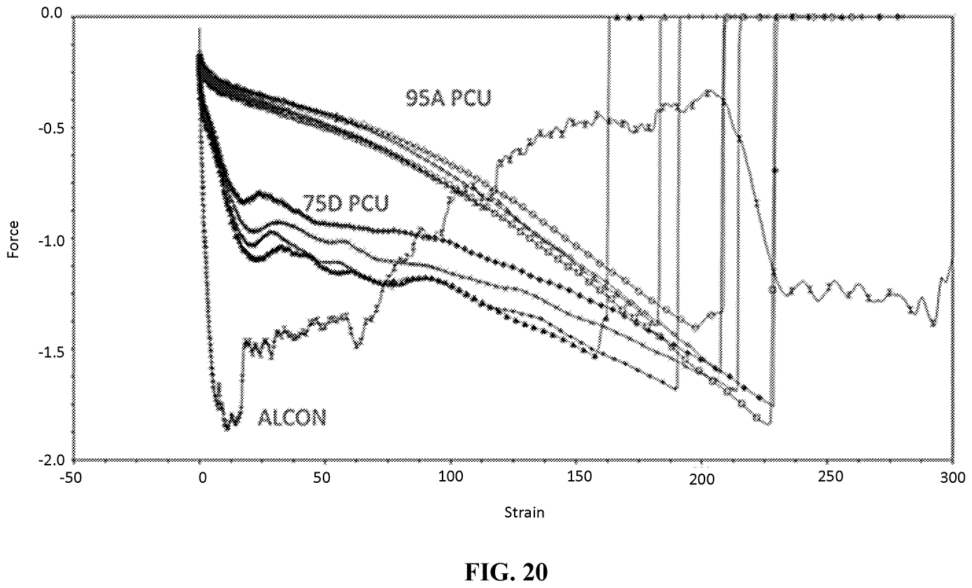

FIG. 20 shows a plot of fatigue and cyclic loading of 3D printed soft polymer for ophthalmic applications. Referring to FIG. 20, it is possible to see a comparison of stress/strain with a variety of fixation devices printed with 95A durometer polycarbonate-urethane (PCU) and 75D durometer PCU as compared to an Alcon REFORM capsular tension ring. As can be seen, fixation devices formed of 95A and 75D PCU are comparable in durability with capsular tension rings on the market.

No admission is made that any reference, including any non-patent or patent document cited in this specification, constitutes prior art. In particular, it will be understood that, unless otherwise stated, reference to any document herein does not constitute an admission that any of the document forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. It is to be understood that the subject matter defined in the appended claims is not necessarily limited to the specific features or acts described above. Rather, the specific features and acts described above are disclosed as examples of implementing the claims and other equivalent features and acts are intended to be within the scope of the claims.

1-18. (canceled)

19. An intraocular lens (IOL) fixation device, comprising:
a semi-rigid frame formed of polymer memory material, the frame having a receptacle for an IOL;
fixation arms connected to and arrayed around the frame; and
a collapsible T-shaped fixation element disposed on a distal end of each fixation arm of the fixation arms, each fixation element having a neutral-stress T-shape.

20. The device of claim 19, wherein the frame is elongated.

21. The device of claim 19, wherein a top of each T-shaped fixation element is protruding and smooth.

22. The device of claim 19, wherein a top of each T-shaped fixation element has a rounded profile.

23. The device of claim 19, wherein a top of each T-shaped end has a disc shape.

24. The device of claim 19, wherein a cross section of each fixation arm of the fixation arms is rectangular.

25. The device of claim 19, further comprising:
an IOL held within the receptacle of the frame.

26. The device of claim 25, wherein a haptic of the IOL is surrounded by the device.

27. The device of claim 26, wherein the haptic of the IOL is held within a lumen of the frame.

28. The device of claim 26, wherein the haptic of the IOL is allowed to expand into a negative space within the frame.

29. The device of claim 25, wherein the device and IOL are collapsed into a linear confined state within a lumen of a cannula, catheter, or needle.

30. The device of claim 29 wherein each T-shaped fixation element is canted to fit within the lumen.

31. The device of claim 19, wherein each fixation element was produced by three-dimensional (3D) printing.

32. The device of claim 19, further comprising:
a biocompatible polymer coating over each fixation element.

33. The device of claim 19, wherein each fixation arm includes a nitinol portion.

34. The device of claim 19, further comprising:
a second plurality of fixation elements, each fixation element of the second plurality of fixation elements located within the frame.

35. The device of claim 19, wherein the frame comprises polymers selected from the group consisting of poly(ethylene terephthalate), polylactide, polyglycolide, polytetrafluoroethylene, poly(vinylidene fluoride), polydimethγl siloxane, and polyetherurethane, polyurethane urea, polyetherurethane urea, polyurethane with carbonate linkages, and polyurethane with siloxane segments.

36. A method of delivering an intraocular lens (IOL) fixation device into an eye, the method comprising:
providing a lumen within a cannula, catheter, or needle into which is collapsed an IOL fixation device, the IOL fixation device comprising a semi-rigid frame formed of polymer memory material, the frame having a receptacle for an IOL, fixation arms connected to and arrayed around the frame, and a collapsible T-shaped fixation element disposed on a distal end of each fixation arm of the fixation arms, each fixation element having a neutral-stress T-shape;
inserting the cannula, catheter, or needle through a sclera wall of an eye;
deploying the IOL fixation device from the lumen to inside the eye such that the device expands to a neutral-stress shape; and
pulling, with a grasper, the T-shaped fixation elements through the sclera.

37. The method of claim 36, further comprising:
allowing, after pulling through the sclera, one or more of the T-shaped fixation elements to open into a respective neutral-stress shape beneath a conjunctiva.

38. The device of claim 36, wherein an IOL is held within the receptacle of the frame.
